(19) United States
(12) Patent Application Publication
(10) Pub. No.: US 2007/0219575 A1
(43) Pub. Date: Sep. 20, 2007

(54) Orthonostric device and method of forming the same

(76) Inventor: Marta L. Mejia, Doral, FL (US)

Correspondence Address: DENNISON, SCHULTZ & MACDONALD, 1727 KING STREET, SUITE 105, ALEXANDRIA, VA, 22314, US

(21) Appl. No.: 11/716,645

(22) Filed: Mar. 12, 2007

Related U.S. Application Data
(60) Provisional application No. 60/783,391, filed on Mar. 20, 2006.

(51) Int. Cl. A61M 29/00

(52) U.S. Cl. 606/196; 606/191

(57) ABSTRACT

There is herein described an orthonostric device in the form of a nasal stent for insertion in the nostril of a patient for the correction of abnormalities and stenosis of the nostril. The stent is formed so that it can be expanded or contracted laterally by means of a tool which permits adjustment without the removal of the stent. A wire retaining clip is affixed to the stent and extends about the alar crease of the nose for retention of the stent. A method of forming the stent is also described which involves taking a casting of the patient's nostril, forming a mold and placing a resin liquid mix therein. Before setting of the resin, a separator or divider is inserted in the mold to separate the stent into two halves which are joined by a jack screw at the proximal end to permit adjustment by means of the tool.

### CLAIM OF PRIORITY UNDER 35 U.S.C. §119(e)

Applicant claims the benefit of priority of Provisional Patent Application No. 60/783,391, filed on Mar. 20, 2006.

### FIELD OF THE INVENTION

The present invention relates to a new medical device to be used for the correction of stenosis of the nostril and to alter the shape of the nostril. The invention also encompasses a new and novel method of manufacture of an orthonostric devise for correction of stenosis of the nostril in a human patient.

### BACKGROUND OF THE INVENTION

Maintaining and correcting the shape and size of the human nostril in patients with cleft lip, cranio-facial abnormalities and the like frequently poses severe problems to the plastic surgeon, post surgery. In many such cases, after surgery a nasal stent of a fixed size dependent on the patient's specific measurements and nostril configuration must be inserted and retained in the nasal cavity.

Previously, round stenting tubes have proven unsuitable, since the normal nostril shape is not truly round. Soft silicone rubber stents are commercially available that have been specifically made for the human nostril shape. Porex® stents are available in the United States and Koken devices are available in Japan.

These prior art stents tend to be easily extruded in use, particularly in children unless they are retained in proper placement with transseptal sutures. Accordingly these devices, even with the use of suture retention, typically are not retained in place in children for longer than a few weeks. Additionally, several cases have been reported where the transseptal sutures have cut through the columella.

It is a primary object of my invention to provide an orthonostric appliance that is easy and relatively inexpensive to manufacture and which provides for lateral expansion and adjustment within the nostril to help correct unaesthetic appearance in patients.

It is a further object of the invention to provide an orthonostric appliance that can be easily removed when desired and which can be worn comfortably by the patient for an extended period of time if required so that the deformity can be properly corrected.

Another object of the invention is to provide an orthonostric appliance that can be adjusted laterally of the nostril axis by means of a jack screw that can be accessed from outside of the nasal cavity by a small elongated tool for purposes of adjustment of the effective width of the nasal cavity without the necessity of removal of the device.

An additional object of the invention is to provide an orthonostric appliance that may be comfortably held in proper position by an adjustable wire flange adopted to wrap around the alar crease of the nostril.

It is a further object of my invention to provide a simple and effective method for forming an orthonostric appliance that accurately conforms to the configuration of the patient's nostril.

Further objects of the invention will become apparent upon a careful reading of the appended specification, claims and drawings, wherein like reference characters refer to the same elements which appear in the several views.

### DESCRIPTION OF RELATED ART

It has been previously suggested and reported in the literature to bend hollow acrylic tubing into a horseshoe shape or to fashion splints from polyvinyl dental-bite registration materials, fabrication of splints from methylmethacrylate, and altering the Koken stents by forming a more everted rim.

Expansible stents, where the stent is sectioned perpendicular to the desired expansion axis have also been described in the prior art (Ramstad, T. and Gorm, B. “Nasal Stenosis after Operations on the Nose: Expansion and Subsequent Maintenance of the Nasal Airway”, Scand. J. Blast. Reconstr. Hand Surg, Scand. J. Plast. Surg., 28, 1994, pp 235-238; Costa, P., Orlando, A. and Di Mascio. “An Expansible Splint for Treatment of Nostril Stenosis”, Ann. Plast. Surg., 34, 1995, pp 197-200; and Cenzi, R. and Guarda, L. “A Dynamic Nostril Splint in the Surgery of the Nasal Tip; Technical Innovation”, J. Craniomaxillofac. Surg. 24:2, 1996, pp 88-91).

In these known constructions, the screw drive for expanding the device is located along the desired axis of expansion and is intranasal, requiring removal of the stent in order to access the screw for adjustment.

In addition to the above, all of the known stents taper down as they pass intranasally creating difficulties in that the stent tends to slide or slip out of the nostril and taping or suturing is then required in order to maintain the stent in place which is cumbersome and often ineffective. Further it creates discomfort and anxiety in the patient, especially children.

### SUMMARY OF THE INVENTION

Consistent and consonant with the present invention, an externally adjustable and expansile nostril stent has been disclosed for correction of abnormal nostril shape and nostril stenosis. The stent may be retained in place by means of a wire retainer or flange which extends from the stent and passes around the alar rim and into the alar crease of the nose.

A method of manufacturing the expansible nostril stent of the invention is also disclosed which is custom-fabricated for each patient and includes making an impression of the nostril of the patient from a plastic substance which is then placed in gypsum. Orthodontic resin powder in a liquid mix is poured into the resulting mold and the adjustment screw and wire retainer are inserted before the resin fully hardens.

### BRIEF DESCRIPTION OF THE DRAWINGS

These and other objects of my invention will be appreciated and understood by those skilled in the art from the detailed description of the preferred embodiment of the invention and from the following drawings in which:

FIG. 1 is a bottom right perspective view of a nostril stent constructed according to my invention and showing the jack screw and wrench tool;

FIG. 2 is a front view of a portion of the face of a typical patient with the stent of the invention inserted into place in a nostril and retained therein;

FIG. 3 is a cross sectional view of the preferred embodiment of the stent of FIG. 1;

FIG. 3A is a cross-sectional view of a second embodiment of the stent of my invention;

FIG. 4 is a front perspective view illustrating in exploded form the retention clip or wire flange for retention of the stent;

FIG. 5 is a perspective view similar to FIG. 4 but showing the positioning of the retention clip within the patient's nostril;

FIG. 6 illustrates the manner of pouring the plastic resin mixture from which the stent is formed into a mold created of the patient's nostril;

FIG. 7 is an exploded perspective view of the mold of FIG. 6 and showing the separator, jack screw and retention clip prior to their insertion in the liquid resin mixture;

FIG. 8 is a perspective view of the mold with all of the elements of FIG. 7 in place; and

FIG. 9 is an exploded view of the hardened stent of FIG. 8 removed from the mold.

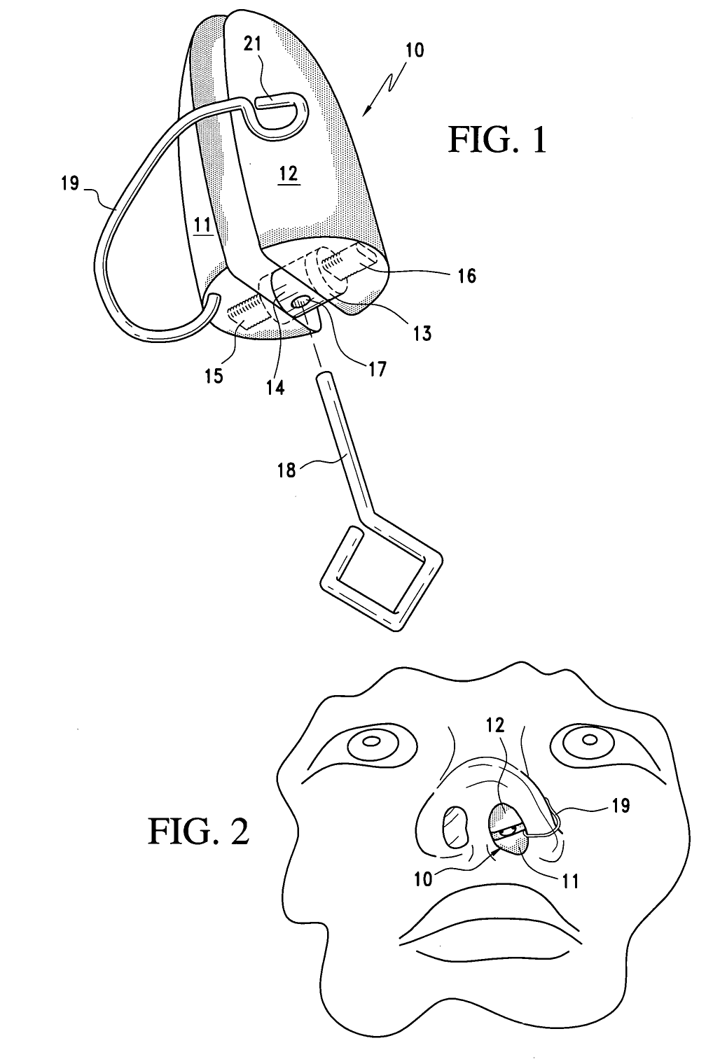

### DETAILED DESCRIPTION OF A PREFERRED EMBODIMENT

With reference to the drawings wherein like reference characters designate like or corresponding elements throughout the several views, it will be seen that the invention comprises an orthonostric device or stent shown generally at 10. The stent is preferably molded from an acrylic orthodontic resin powder and liquid as more fully described later herein and has a generally conical shape.

In the preferred embodiment, the stent 10 is formed with two generally identical portions 11 and 12, which together are formed to fit within the nostril of the patient and conform to the internal shape thereof.

The two stent portions 11 and 12 are held together by a jack screw 13 which comprises a barrel 14 and two oppositely threaded shafts 15 and 16. It will be appreciated that the extending portions of the threaded shafts are embedded within the stent portions 11 and 12. A plurality of transverse openings 17 are formed within the barrel 14 to receive a jack wrench or turning tool 18 therein.

It should be understood that once the stent is in place, the jack screw can be rotated by means of tool 18 in order to move the two portions of the stent 11 and 12 toward or away from each other for the purpose of exerting pressure on the nostril walls to gradually conform the same to normal shape. It will be noted that it is unnecessary to remove the stent for such adjustment.

A retaining clip or wire flange 19 formed from stainless steel and having a diameter of approximately 0.9 mm extends from the molded stent. The wire is formed with a series of bends 20 at its proximal end in order to anchor the same firmly within the stent while it sets. The distal end curves upwardly as shown and terminates in a small loop or hook 21 to assist in stent placement and to avoid a sharp end which could cut or damage the patient or surgeon.

The retaining clip is so formed so as to gently hug the alar crease of the nose and extends about the alar rim in order to hold the stent comfortably in place and prevent premature expulsion or withdrawal.

Proper fit and retention is very important since the orthonostric device may be worn for many months both at night and during the day.

The stent described above is of primary importance in restoring the nostril size and shape, especially for patients with cleft lip or crania-facial anomalies. With conventional nasal stents, the same are used for no more than one week post surgery and in many cases, the nasal tissue may collapse and will not permit optimal esthetic results.

In order to custom-form the stent of my invention, an impression of the patient's nose is taken using any orthodontic or similar mold casting material. The resulting mold 22 serves as a reservoir 23 into which a mixture of orthodontic resin powder and liquid 24 may be poured from a container 25 as shown in FIG. 6.

Before the resin sets, a separator member 26 is inserted in the mold and also serves to carry and set in place the jack screw 13. The separator divides the stent into the two portions 11 and 12. Also during the setting of the resin, the retaining clip 19 is inserted within the resin. The entire assembly is removed from the mold as shown in FIG. 9 and then the separator may be removed so that the two portions 10 and 11 are held together solely by the jack screw 13.

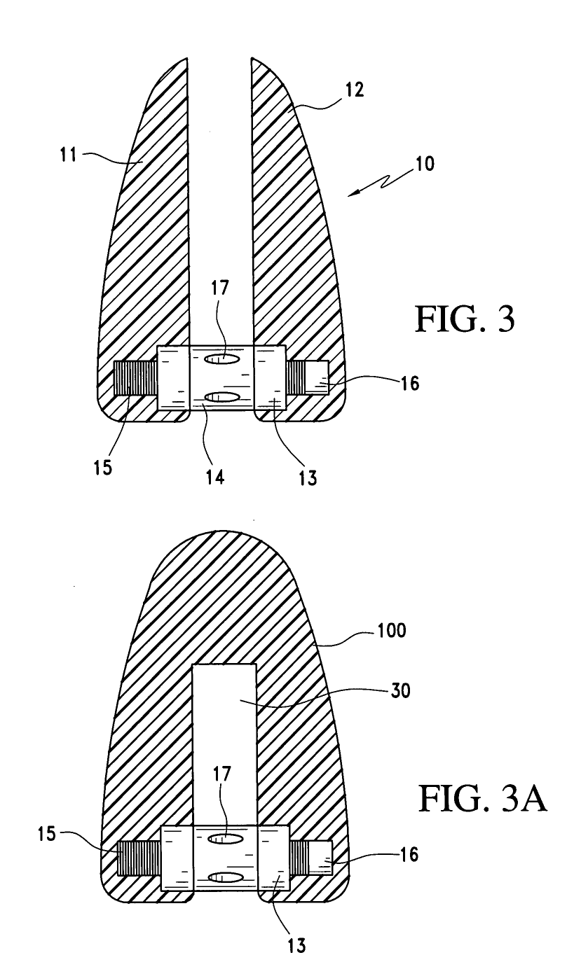

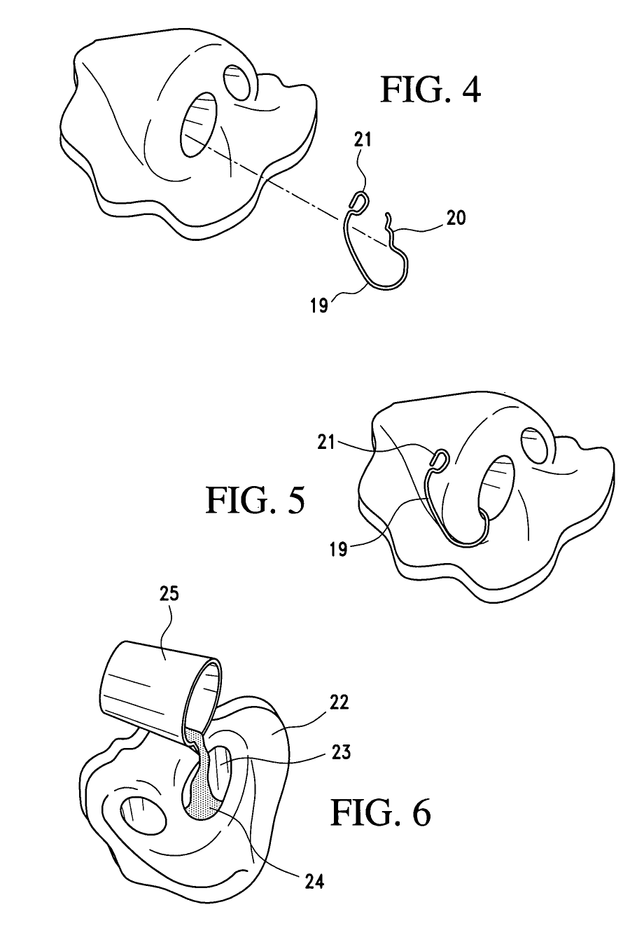

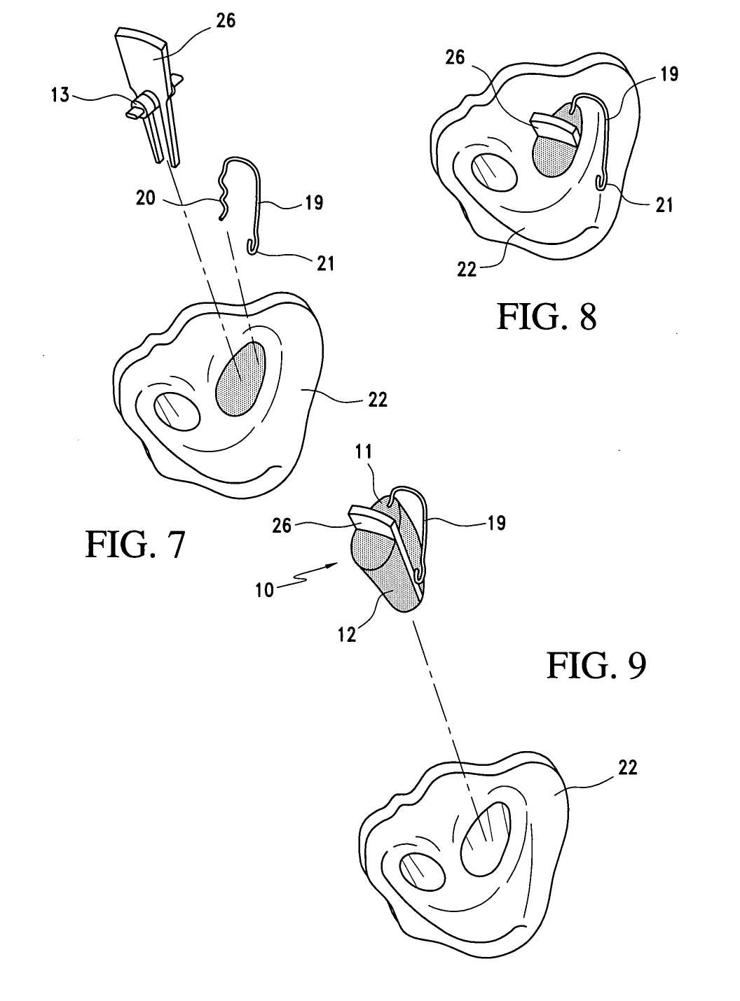

In a second embodiment of the invention as shown in FIG. 3A, a shorter separator is employed in casting the stent and therefor the resulting stent is a single piece 100 and does not include two portions. It does have a slot 30 extending vertically from the open end slightly more than half of the length of the stent. The resilience of the stent material will in such case permit limited adjustment by the jack screw in the nostril of the patient.

It is contemplated that two stents can be formed for insertion in both nostrils of the patient in certain cases.

I claim:

1. An orthnostric device in the form of a generally conical stent for insertion within the nostril of a patient, said stent comprising two spaced apart elongated sections, said sections being joined together at their proximal ends by jack means for moving the said sections together or apart for purposes of adjustment within the nostril.

2. The device of claim 1, wherein the jack means comprises a barrel having oppositely threaded elongated screw shafts extending from the lateral ends thereof, said shafts being fixed in said elongated sections.

3. The device of claim 2, wherein said barrel of the jack means is provided with a plurality of tool-receiving openings about the periphery thereof, and an elongated tool for insertion into one of said openings for turning the barrel in order to move said sections inwardly or outwardly.

4. The device of claim 1, wherein the stent is formed of a plastic material custom configured to fit within the nostril of a patient.

5. The device of claim 1, and further including a retaining clip fixed in the stent and extending outwardly therefrom so as to curve out and upwardly around the alar rim of the patient's nostril and being formed to fit within the alar crease of the nose in order to secure the stent in place.

6. The device according to claim 5, wherein the retaining clip is formed with a curved hook at its free end to assist in handling the stent.

7. The device according to claim 3, and further including a retaining clip fixed in the stent and extending outwardly therefrom so as to curve out and upwardly around the alar rim of the patient's nostril and being formed to fit within the alar crease of the nose in order to secure the stent in place.

8. An orthnostric device in the form of a generally conical stent for insertion within the nostril of a patient, said stent comprising a custom-formed conical configuration having an elongated slot extending inwardly from the distal end thereof dividing the stent into two spaced apart elongated sections on either side of said slot, said sections being joined together at their ends by jack means for moving the said sections together or apart for purposes of adjustment within the nostril.

9. The device of claim 8 and further including a retaining clip fixed in the stent and extending outwardly therefrom so as to curve out and upwardly around the alar rim of the patient's nostril and being formed to fit within the alar crease of the nose in order to secure the stent in place.

10. The device of claim 8 wherein said jack means includes a barrel provided with a plurality of tool-receiving openings about the periphery thereof, and an elongated tool for insertion into one of said openings for turning the barrel in order to move said sections inwardly or outwardly.

11. A method of forming a nasal stent including the steps of:
a. making a plaster impression of the nasal cavity of a patient;
b. forming a mold from the said impression;
c. placing a liquid resin mix within the nasal cavity of the mold;
d. inserting a vertical spacer within the mold before the liquid resin mix sets in order to divide the stent into two portions;
e. placing a jack screw means within the mold before it sets, said jack screw means having laterally extending screw shafts which are received in the two portions; and
f. removing the vertical spacer.

12. The method of forming a nasal stent as defined in claim 10 and further including:
insertion of a curved retainer clip within the liquid resin mix before it sets, which clip extends outwardly of the mold.

13. The method of claim 11, wherein the resin is an orthodontic resin powder.
